(19) United States
(12) Patent Application Publication
(10) Pub. No.: US 2003/0175784 A1
(43) Pub. Date: Sep. 18, 2003

(54) Method for detecting, analyzing, and mapping RNA transcripts

(75) Inventors: Jeffrey J. Leary, Wayne, PA (US); Ruth Tal-Singer, Media, PA (US)

Correspondence Address: GLAXOSMITHKLINE, Corporate Intellectual Property - UW 2220, P.O. Box 1539, King of Prussia, PA, 19406-0939, US

(73) Assignee: SmithKline Beecham Corporation

(21) Appl. No.: 10/388,281

(22) Filed: Mar. 13, 2003

Related U.S. Application Data
(63) Continuation of application No. 09/719,714, filed on Dec. 15, 2000, now abandoned.
Application No. 09/719,714 is a 371 of international application No. PCT/US99/13813, filed on Jun. 18, 1999.
(60) Provisional application No. 60/090,464, filed on Jun. 24, 1998.

(51) Int. Cl. C12Q 1/70; C12Q 1/68; G06F 19/00; G01N 33/48; G01N 33/50

(52) U.S. Cl. 435/6; 702/20; 435/5

(57) ABSTRACT

A genetic analysis method termed “fine array transcript mapping” or “FAT Mapping” is disclosed, which method is useful for detecting and measuring RNA molecules, which have been transcribed from a genome. The method can be applied to explore differential expression of a template genome, and for accurately mapping the 5′ ends of transcripts, which have been expressed. Further, the presence or absence in any particular biological circumstances of a given transcript and its relative concentration can define gene functions or coding capacities. Thus the method relates to mapping and identifying novel and known gene products and investigating gene functions and regulation.

### CROSS REFERENCE TO RELATED APPLICATIONS

This application claims priority to U.S. Ser. No. 60/090,464 filed Jun. 24, 1998, which is incorporated by reference in its entirety.

### FIELD OF THE INVENTION

This invention relates to a novel genetic analysis method, fine array transcript mapping, or “FAT Mapping”, which is a method useful for detecting, measuring, and characterizing RNA molecules which are transcribed from a genome. The method is especially useful for determining the differential expression of RNAs between two samples and for accurately determining the ends of the RNA molecules (mapping) with respect to a template, genomic sequence.

### BACKGROUND OF THE INVENTION

The analysis of transcriptional regulation of complex genomes is an experimental challenge. One classical approach has employed filter hybridization, or northern blotting, which analyzes transcripts from only one small region of a genome at a time; that portion represented by the probe. Complete transcriptional analysis of complex genomes by this technique requires hundreds or thousands of experiments and a daunting amount of time and effort. Further, each biological circumstance investigated necessitates an additional, separate analysis of the genome. Thus, this traditional approach has significant drawbacks in terms of efficiency.

To overcome these drawbacks, increasingly sophisticated and sensitive approaches have been developed which rely upon reverse transcriptase-polymerase chain reaction (RT-PCR) to demonstrate expression of specific genes in different cell populations. Differential display RT-PCR (DDRT-PCR), the first of these newer PCR-based methods, employs random-primed amplification of total mRNA from two populations. DDRT-PCR allows the visualization and subsequent isolation of cDNA fragments corresponding to mRNAs which display altered expression in the two RNA populations (7, 8). Another method, termed representational difference analysis (RDA), is a process of subtraction of fragments present in two populations which is coupled to amplification of cDNA fragments from differentially expressed mRNAs present in one of the populations (6, 9). A third method, called suppression subtractive hybridization (SSH) uses RT-PCR to selectively amplify mRNAs from differentially expressed genes while suppressing amplification of abundant cDNA's (2). In a recent study the present inventors employed DDRT-PCR to isolate 32 differentially-displayed mouse cDNAs representing transcripts whose levels were altered within the first 4 hours following explanation of latently HSV-1-infected murine trigeminal ganglia. It was found that four cDNAs were identical to murine TIS7, whose sequence has been shown to be related to interferons (IFNs) (15). The processing of this experiment took approximately one year to accomplish. The acrylamide gel purification, re-amplification, confirmation, and sequencing of each differentially expressed fragment produced by DDRT-PCR was a very labor-intensive process.

Once a portion of an mRNA sequence is identified by DDRT-PCR, RDA or SSH, the protein encoding portion of the RNA can be determined only after the true ends of the transcript are mapped. Sophisticated methods for accomplishing the mapping of the ends of a few mRNA's sharing a known sequence in one batch have also been developed. Preeminent among these is the method known as “rapid amplification of cDNA ends” (RACE) or “one-sided amplification”, which is applied to 3′ ends or 5′ ends separately (18,19,20,21). This procedure uses one oligonucleotide primer comprising a sequence known to be expressed in an mRNA and a second generic oligonucleotide primer characteristic of the ends of mRNAs. Only a small set of RNA molecules, all originating from the genomic region containing the sequence represented by the first oligonucleotide primer, can be detected or analyzed in one experiment.

The present invention, termed “fine array transcriptional mapping” or “FAT Mapping” is yet a further development in this area. FAT Mapping involves probing a test grid containing an array of hundreds to thousands of overlapping genomic clones or DNA fragments with probes consisting of labeled cDNAs representing the RNA transcripts from test populations (1, 11, 12). Preferably using high-speed robotics, this potentially high capacity system allows quantitative measurements of the expression of rare transcripts from probe mixtures derived from microgram amounts of total cellular mRNA, and enables the analysis of hundreds of genes within a genomic sequence in a single run. Recently, using a similar technique, oligonucleotide arrays have been used to identify novel open reading frames (“ORFs”) in yeast (16). Because of the large number of clones employed in the FAT Mapping technique resulting in short gaps between the ends of any two adjacent clones, the ends of labeled probes can be predicted with a high degree of accuracy. Preferably, the accuracy of the prediction is proportional to the number and distribution of the clones in the array. The accuracy can be predicted by computer simulation. Thus, FAT Mapping is a technique capable of accomplishing the goals of DDRT-PCR, SSH, RDA and RACE in a very rapid, labor saving manner. The FAT Mapping process can also be used to complement and confirm studies which utilize art-recognized methods to identify differentially expressed gene sequences and to map transcripts. Furthermore, FAT Mapping allows the generation of a database of induced, differentially expressed genes from a single experiment which will facilitate the identification of previously unknown regulatory elements in transcriptional promoters common to those expressed genes.

Previously unidentified genes may also be located within a given genomic sequence using the FAT Mapping method. The genomes of viruses, particularly herpes viruses, represent one example of genomic sequences to which the present FAT Mapping method can be advantageously applied. For example, it is known that gene activity and transcription of genes in herpes simplex virus type 1 (HSV-1) is temporally regulated in a cascade during infection of cultured cells in vitro. It is further known that herpes viruses express different proteins from transcripts which have common 3′ ends but different 5′ ends. For example, in previous studies, Bandaran et al. (17) described the identification of a new protein, OBPC, encoded by herpes simplex type 1 which was discovered by accurately determining the 5′ end of mRNA's containing the UL9 open reading frame by more classical methods. The OBPC protein was encoded by a novel transcript (UL8.5) with a different 5′ end, but the same 3′ end, as the UL9 transcript encoding the OBP protein. Thus, it is clear from these results that mapping the ends of RNA transcripts is a method of discovering new genes, although using traditional techniques the discovery of new genes in this way is very labor-intensive. FAT Mapping provides a novel and rapid method of globally mapping the ends of transcripts within large genomic regions at once, and therefore the method of the invention provides an alternative very efficient method enabling the discovery of previously unidentified genes.

### SUMMARY OF THE INVENTION

The present invention provides a method of mapping the position of an individual transcript from a genomic sequence, comprising the steps of: a) generating overlapping subfragments of the genomic sequence, wherein at least a portion the nucleotide sequence of each genomic subfragment has been determined; b) placing each overlapping genomic subfragment in a separate ordered (known) position on a high density grid; c) preparing a composition comprising test transcripts which have been transcribed from said genomic sequence; d) labeling the test transcripts in said composition in a detectable manner; e) placing the composition comprising the labeled test transcripts in contact with the high density grid containing the genomic subfragments, whereby the labeled test transcripts are allowed to hybridize to the genomic subfragments; f) removing unhybridized test transcripts from the surface of the high density grid; g) detecting on the high density grid the ordered positions which contain a hybridized labeled test transcript; and h) analyzing the pattern in which the labeled test transcripts have hybridized to the genomic subfragments on the high density grid, whereby by comparing the position of the labeled test transcripts on the high density grid to the ordered position of the overlapping genomic subfragments on said grid, the position of individual test transcripts from within the genomic sequence are mapped.

The invention also provides a method of measuring the differential expression of transcripts between two or more different tissue or cell populations which share a common genomic sequence, comprising conducting the above described steps a. and b. on said common genomic sequence; separately performing the above described steps c. through h. on each different tissue or cell population; and comparing the pattern in which the test transcripts from each different cell or tissue population have been mapped to the common genomic sequence, whereby differences in the expression of transcripts between the different tissue or cell populations is determined.

The present invention further provides a method of determining whether a particular open reading frame of known position within a genomic sequence is expressed under particular conditions, comprising the steps of conducting above described steps a. and b. on a genomic sequence, whereby the ordered position on the high density grid of genomic subfragments corresponding to said particular open reading frame is determined; subjecting a population of cells or tissues containing said genomic sequence to a particular condition; conducting above described steps c. through h. on the genomic sequence of said cells or tissues which have been subjected to the particular condition; and determining whether test transcripts from said cells or tissues which have been subjected to said particular condition have hybridized to the ordered positions on said high density grid corresponding the genomic subfragments of said particular open reading frame, whereby it is determined whether said open reading within said genomic sequence has been expressed under said particular condition.

### BRIEF DESCRIPTION OF THE DRAWINGS

FIG. 1 illustrates the fine array transcript mapping, or “FAT Mapping” process applied to a single genome, the genome of herpes simplex virus type 2 (HSV-2). The HSV-2 genome is an example of a large, transcriptionally complex genomic region. Over 2,000 random, overlapping clones of the HSV-2 DNA genome were generated and the cloned DNA fragments were sequenced at each end. Each individual cloned fragment is placed on an individual spot in an array on a gridding medium, for example nylon membrane or a glass slide. On average, every nucleotide in the HSV-2 genome is represented in several of the clones on the array.

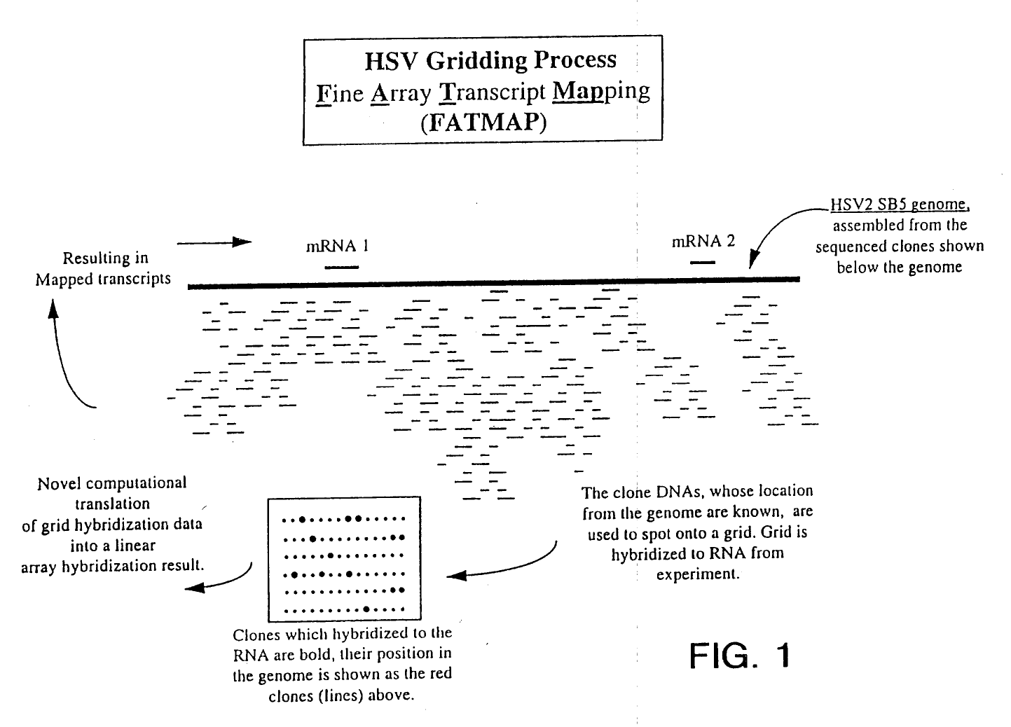

FIG. 2 depicts a complexity of transcripts from the internal repeat region of HSV-1 as mapped by conventional methods.

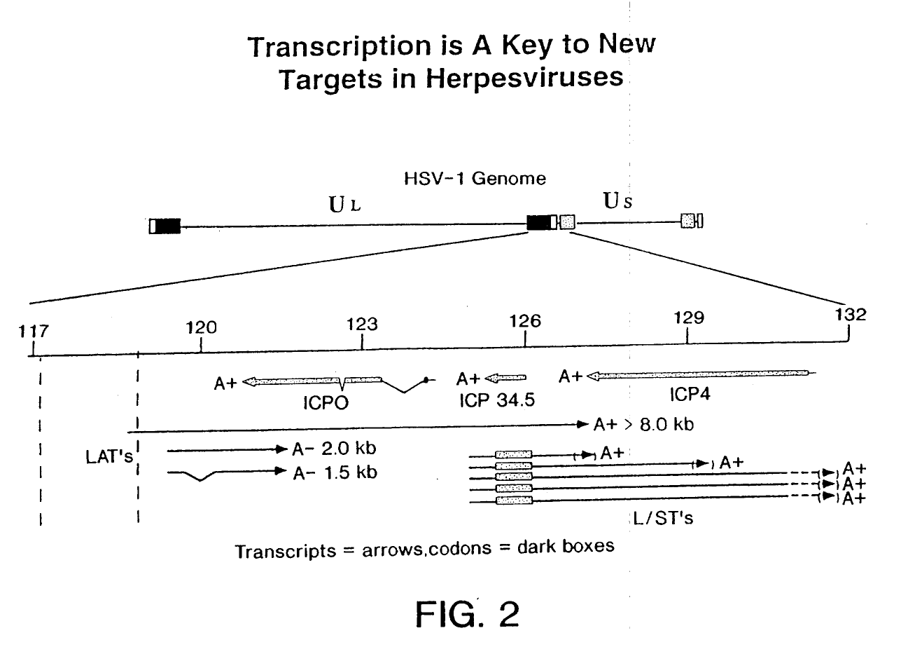

FIG. 3A depicts the results of hybridizing FATMap arrays with cDNA probes prepared from MRC-5 cells infected for 0, 2, 6 and 17 hours. The genomic location of the left end of all subfragment clones from between HSV-2 genome nucleotides 87000 and 91000 is used as the X-coordinate, while the height of each symbol on the Y-axis is the light intensity of the grid spot that the subfragment occupied.

FIG. 3B depicts the HSV-2 ORFs located between genome nucleotides 87000 and 91000 predicted from the genbank entry for HSV2HG52, described in the features section of the genbank entry and drawn with the software package MapDraw (DNASTAR, Inc.). UL39 (ICP6) is the only known ORF in this genomic region.

FIG. 4 depicts the grid hybridization results for PCR products generated specifically for testing the expression of the UL39 ORF alone after hybridization with cDNA probes prepared from HSV-2 infected MRC-5 cells at 0, 2, 6, and 17 hours PI. This represents the conventional approach to microarray analysis as opposed to FAT Mapping. The product was spotted onto 5 separate locations on each grid, resulting in data from spots 1 to 5.

FIG. 5A represents the results of conventional semi-quantitative RT-PCR analysis of ICP6 mRNA amounts by comparison with the amounts of mRNA for the house-keeping gene beta-actin. The ratios of the amount ICP6 gene-specific PCR product to that for beta-actin calculated from RT-PCR reactions on RNA from HSV-2 infected MRC-5 cells at 0, 1, 2, 4, and 6 hours PI are shown.

FIG. 5B depicts the relative amount (copy number) of mRNA molecules detected by quantitative TaqMan PCR in RNA samples from HSV-2 infected MRC-5 cells at 0, 1, 2, 4, and 6 hours PI. Transcripts for HSV-2 genes gC (UIA4), IPC6 (UL39) and ICP27 were measured and are shown in the bar graph.

FIG. 6A depicts the results of hybridizing FATMap arrays with cDNA probes prepared from MRC-5 cells infected for 0, 2, 6 and 17 hours. The genomic location of the left end of all subfragment clones from between HSV-2 genome nucleotides 96000 and 101000 is used as the X-coordinate, while the height of each symbol on the Y-axis is the light intensity of the grid spot that the subfragment occupied. The signal intensity of all clones in the region of UL44 (gC) between 97000 and 98000 increases from 0 to 2 and from 2 to 6 hours PI, and then decreases slightly at 17 hours PI.

FIG. 6B depicts the HSV-2 ORFs located between genome nucleotides 96000 and 101000 predicted from the genbank entry for HSV2HG52, described in the features section of the genbank entry and drawn with the software package MapDraw (DNASTAR, Inc.). UL44 (gC), UL45 and portions of UL43 and UL46 are the known ORFs in this genomic region.

FIG. 7 depicts the results of conventional microarray gene-specific PCR product spots for the UL44 open reading frame hybridized to cDNA probes prepared from MRC-5 cells infected for 0, 2, 6 and 17 hours. The gene-specific DNA was put on 8 replicate spots in the microarray.

FIG. 8 depicts both the results of hybridizing FATMap arrays with cDNA probes prepared from MRC-5 cells infected for 0, 2, 6 and 17 hours and known ORFs drawn from the HSV2HG52 genbank entry with MapDraw. The genomic locations of the left end (filled symbols) and right end (open symbols) of all subfragment clones from between HSV-2 genome nucleotides 58000 and 64000 are used as the X-coordinate, while the height of each symbol on the Y-axis is the light intensity of the grid spot that the subfragment occupied. UL29 is the only known gene predicted from the HSV2HG52 sequence entry between genome nucleotide numbers 58000 and 64000.

FIG. 9 depicts both the results of hybridizing FATMap arrays with cDNA probes prepared from MRC-5 cells infected for 0, 2, 6 and 17 hours and known ORFs drawn from the HSV2HG52 genbank entry with MapDraw. The genomic locations of the left end (filled symbols) and right end (open symbols) of all subfragment clones from between HSV-2 genome nucleotides 22000 and 28000 are used as the X-coordinate, while the height of each symbol on the Y-axis is the light intensity of the grid spot that the subfragment occupied. Signal intensity in this genome region correlates well with known ORFs, where UL9 and UL13 are for instance expressed only at low levels while UL10 and 11 are rather highly expressed in contrast to the pattern seen with the UL29 region depicted in FIG. 8.

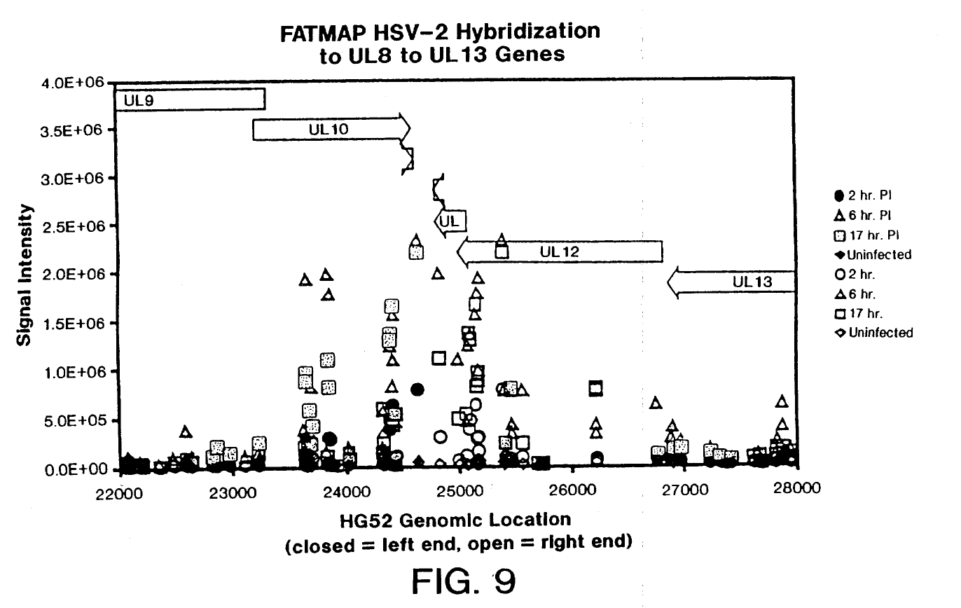

### DETAILED DESCRIPTION OF THE INVENTION

The present FAT Mapping invention provides a convenient method of mapping the position within a given genomic sequence of any individual transcript which has been expressed from that genomic sequence. The general method comprises the steps of first generating overlapping subfragments of the genomic sequence, wherein the nucleotide sequence of each subfragment has been determined or is known. Regarding this step of the process the term “sequenced” does not necessarily entail determining the entire nucleotide sequence across each genomic subfragment. Specifically it is often sufficient to know only enough of the sequence, for example, at each end of the fragment (5′ and 3′ ends) to be able to determine the position within the genomic sequence from which that subfragment has been derived. Further, in some cases, if the degree of overlap of the subfragments is extensive, it may be sufficient to sequence only a substantial portion from one of the ends (5′ or 3) of each subfragment. With respect to this step of the invention, the purpose of determining all or some of the sequence of the subfragments is simply to be able to determine the correct order of those subfragments across the genomic sequence.

Sequencing of the genomic subfragments may be accomplished by any convenient methodology, of which several are well known in this art. Also, in a particularly preferred embodiment of this step, the individual subfragments are amplified using, for example the polymerase chain reaction, prior to sequencing or prior to placement of the subfragments onto the high density grid.

Once the genomic subfragments of known sequence have been generated, aliquots of each subfragment are placed individually in an ordered (known) position onto a high density grid. Since the position of each fragment on the grid is known, and the location of each fragment's sequence in the whole genomic sequence is known, then the data resulting from any grid position can be assigned to the small region of the genomic sequence represented by the subfragment. For purposes of this method, the term grid, or high density grid, refers to any surface which is suitable for receiving ordered spots or aliquots of genomic subfragments. Nucleic acid grid materials include, for example, nylon filter membranes, derivatized glass, silicon chips or other polymeric solid supports. Many such grids are commercially available.

The grid loaded with aliquots of genomic subfragments is then exposed to a composition comprising test transcripts which have been transcribed from cells or tissues containing the genomic sequence. The test transcripts have been prepared to be labeled in a detectable manner. Methods of detectably labeling test transcripts include, for example, reverse transcription and polymerase chain reaction in the presence of labeled nucleotide triphosphates. Preferred labels include fluorophores such as flourescein, rhodamine and pyrenes, haptens, P32, P33 ,terbium, europium, and electrically active moieties.

The labeled test transcripts are placed in contact with the high density grid containing the genomic subfragments and are allowed to hybridize to the genomic subfragments. Preferred hybridization conditions include salt concentrations of 0.01 to 1.0M, temperatures of about 35 to 70 degree C., and times of approximately 0.5 to several hours. Preferred conditions are easily determined empirically by those skilled in this art and differ, for example, based upon the average G+C content of the arrayed nucleotides. Unhybridized test transcripts are removed from the surface of the high density grid by any convenient method known in this art. Generally useful methods known in this art for preparing arrays, labeled probes and hybridization conditions are provided, for example, in references 11 and 12.

Next, each ordered position of the high density grid having a labeled test transcript is detected and the pattern in which the labeled test transcripts appear on the high density grid is analyzed, whereby by comparing the position of the labeled transcripts on the high density grid to the ordered position of the overlapping genomic subfragments on said grid, the position of the individual test transcript within the genomic sequence is mapped.

The invention thus conveniently is able to provide accurate localization of the 5′ end of the RNA which has been transcribed, in addition to the 3′ end, thus providing a means of mapping known and unknown transcripts containing ORFs, or genes, onto the genomic sequence. This information is not provided by other hybridization array methods known in the art. Expressed RNAs may possibly contain ORFs which were previously not expected to be actual genes (new genes), and the invention is further capable of associating these ORFs with expression in response to particular conditions or stimuli, and thus information about the function of novel genes is also provided by the invention. It is also possible that information about expression of known ORFs in response to particular conditions or stimuli provided by the method of the invention may lead to identification of a new function or activity for known ORFs. The identification of new genes may include wholly new genes whose sequence and expression has never been characterized, and also new ORFs within known gene sequences wherein transcription initiation takes place at a newly recognized place. The template genomic sequence of interest can be single-stranded or double-stranded DNA or in some cases RNA, derived from any living organism including animal, microbial, viral or plant. Preferred embodiments of this method include wherein the genomic sequence is derived from an animal, particularly a mammal, most particularly a human animal. Further preferred genomic sequences are derived from viruses or bacteria, most particularly herpes simplex viruses type 1 and type 2, hepatitis B virus, hepatitis C virus, human herpes viruses 6, 7, and 8 and other complex genomes such as human cytomegalovirus. Further preferred genomic sequences can be derived from, for example, Pseudomonas artificial chromosomes (BACs) containing genomic regions of other prokaryotic or eukaryotic pathogens or animals, or even complete genomes of Streptococcus sp., Staphylococcus sp., Mycobacterium sp. and other similar organisms which present pathogenic risk to mammals including humans.

Other preferred embodiments of the general FAT Mapping method include wherein the overlapping subfragments are generated by shotgun cloning techniques wherein the DNA of interest is either sheared or digested enzymatically and enough random fragments are cloned such that all sequences of the region are represented by multiple clones. The total population of clones thus represents a library for the genomic region. As mentioned above, in this aspect the cloned fragments may be individually amplified and separated from the cloning vector by using the polymerase chain reaction prior to placing them onto the high density grid. Further, if PCR is used to generate defined overlapping DNA fragments from a genomic region of n nucleotides for FAT Mapping, the fragments are preferably prepared so as to be offset in sequence by few bases, preferably one. Thus, for example, the fragment series will contain fragments of polynucleotides having the sequence base #1 to 200, 2 to 201, 3 to 202, etc . . . (n-199) to n. In a final preferred method of generating the DNA fragments for FAT Mapping, one could completely synthesize an overlapping series of oligonucleotides of 20 or more bases in length from a previously known genomic sequence representing the genome of n bases, such that the series contains oligonucleotides of sequence base #1 to 20, 2 to 21, 3 to 22, . . . (n-19) to n.

Further preferred embodiments of the general FAT Mapping method include employing computer-assisted methods to analyze the positioning of the genomic subfragments over the length of the genomic sequence based upon sequencing data of the genomic subfragments. Further, computer-assisted methods are useful to detect and compare the pattern of the labeled test transcripts on the high density grid to the ordered position of the overlapping genomic subfragments, and also to predict characteristics of the mRNAs and genes they represent through such analysis. Automated steps may be employed at any point of the method to improve efficiency of the method, particularly at steps involving, for example, sequencing of the subfragments, amplification of the subfragments, placement of aliquots of the subfragments or labeled test transcripts onto the high density grid, and in the hybridization and washing steps.

Further provided by the present FAT Mapping invention is a method of measuring the differential expression and relative concentrations of transcripts between two or more different tissues, cell populations or viral-infected cell populations which share a common genomic sequence. This method first comprises, as described above, preparing a high density grid of sequenced, overlapping subfragments of the common genomic sequence. Compositions of test transcripts are then prepared from the common genomic sequence, wherein each test composition represents expression of the common genomic sequence from a different tissue or cell population, or from the same tissue or cell population at a different time point, or from the same tissue or cell population which has been exposed to a specific stimulus or condition. Finally, the pattern of test transcripts expressed from the common genomic sequence in each instance is compared, whereby differences in the expression of transcripts between different tissue or cell populations, or between the same tissue or cell population at different time points, or between the same tissue or cell populations subjected to different stimuli or condition, are determined.

Preferred embodiments in this aspect of the method include wherein the common genomic sequence is derived from a mammal, most particularly a human. Also preferred would be from a bacterial species, most particularly a human pathogen such as Streptococcus, Staphylococcus, Mycobacterium, or a fungus, most particularly a human pathogen fungal type such as Cryptococcus; or a parasitic animal, particularly a eukaryotic human pathogen such as Plasmodium. Especially preferred would be genomic sequences derived from a virus, most particularly a herpes simplex type 1 or herpes simplex type 2 virus.

Further preferred embodiments of this aspect of the FAT Mapping method aimed at analyzing differential expression include wherein test transcript compositions are derived from different tissue types within the same organism, for example when samples are taken from different organs or cell types within an individual animal, particularly a mammal, particularly a human. For this aspect the invention provides a convenient mechanism for investigating regulation of tissue and cell specific function.

The general method further provides a way to investigate expression of the same tissue type at different time points of genomic expression; for example, genomic expression could be measured at different stages of tissue, cellular or viral development, or at different time points after exposure to a particular stimulus or condition. Examples of different time point analyses might include investigation of cellular development and differentiation of higher animals, for example in humans, analysis of fetal tissues compared to the same tissues throughout the aging process. Further particularly useful aspects include analysis of a viral genome within viral-infected cells at different stages of viral genomic expression, for example the viral genome is sampled throughout latency and at intervals during virulence cycles. Accordingly, analysis of a cellular genome could also be performed to investigate the expression of cellular factors in tissues which harbor viruses at various time points associated with viral latency and infection.

The method is also applicable to time point analysis in various tissue and cell types after exposure to a particular stimulus or condition, whereby the effect of that stimulus or condition upon cellular or viral expression is studied. Examples of possible stimuli to different genomic samples are limitless, and include, for example, temperature, light, pressure, or any other physical, environmental or chemical stimuli including particularly chemical compounds, most preferably potential drug candidate compounds which can be exposed to any viral, cell or tissue type in a state of infection or disease. Thus, the present invention provides a useful analytical method of investigating the effect of potential drug candidate compounds on disease states, including classical noninfectious diseases such as cancer tissues, and also including infectious disease states such as viral infection.

The FAT Mapping invention can further be described in yet another aspect, as a method of determining whether a particular open reading frame of known position within a genomic sequence is expressed under any particular time point or condition. The general method, as described above, comprises the steps of generating overlapping subfragments of a genomic sequence, sequencing these subfragments, and placing an aliquot of each sequenced subfragment onto a high density grid in ordered positions. Then, a population of cells or tissue containing this genomic sequence is subjected to a particular condition or sampled at a particular time point, and a composition comprising test transcripts expressed while the viral, cell or tissue population was subjected to the particular condition or time point is prepared. The test transcripts in this composition are detectably labeled and placed in contact with the high density grid, whereby the labeled test transcripts are allowed to hybridize to the genomic subfragments on the grid. Unhybridized test transcripts are washed from the grid, and positions on the grid containing labeled test transcripts are identified. The pattern in which the test transcripts have hybridized to the genomic subfragments on the grid is analyzed, preferably by computer assisted methods. This analysis maps the position(s) on the genomic sequence from which test transcripts have been transcribed, and it is conveniently determined whether a particular transcript from a known open reading frame has been expressed.

A particularly preferred aspect comprises subjecting a tissue or cell population to a particular stress or to a potential drug compound, and determining whether the exposure to the stress or potential drug has stimulated or inhibited transcription from a particular open reading frame of interest.

### EXAMPLES

The following Examples are provided as a means of illustrating various aspects of applicants' invention and should not be construed as limiting the applicability of the general FAT Mapping invention. The Examples as provided refer to and utilize conventional molecular biology and virology techniques which are well-known in these arts, such as those described in Current Protocols in Molecular Biology, Vols. 1 and 2, John Wiley & Sons, 1989 and subsequent updates, which are hereby incorporated by reference into the disclosure of this invention.

General Methods

Preparation of HSV-2 Cloned DNA Specimens for Making the Array: Single bacterial colonies from HSV-2 SB5 (ATCC VR 2546) genomic libraries were selected to ensure unique plasmid insert. Colonies were grown overnight in 175 ul LB broth containing ampicillin in microtiter plates without shaking at 37C. 1 ul culture was used per triplicate PCR amplification wells in 50 ul containing M13 universal primers (Gibco Life Technologies) and AmpliTaq Gold PE.

Amplification proceeded for 40 cycles at 55 degrees C. Products were analyzed by agarose electrophoresis, purified using AGTC columns. DNA was quantitaed, sequenced with M13 universal primer (ABI sequencer) and precipitated for gridding. Bacterial cultures were frozen in triplicates. Gene specific PCR products for controls were generated from genomicHSV-2 SB5 DNA as described below (primer sensitivity).

Microarray Preparation from HSV-2 Cloned DNA: DNA template products from the above step were used to prepare arrays of DNA spots for hybridization. Arrays were spotted on silane treated glass (Molecular Dynamics, Sunnyvale, Calif.) using the Molecular Dynamics Microarray spotter. The protocols used for spotting and hybridization were essentially those described elsewhere (in A Systems Approach To Fabricating And Analyzing DNA Microarrays (1999). Jennifer Worley, Kate Bechtol, Sharron Penn, David Roach, David Hancel, Mary Trounstine, and David Barker. DNA Microarrays: Biology and Technology. Biotechniques Books. Editor Mark Schena). All resulting microarrays were scanned with the Molecular Dynamics microarray scanner after hybridization of cDNA probes prepared as described below. Images were analyzed using ArrayVision (Imaging Research, St. Catherine's, Ontario, Canada).

Extraction of RNA from HSV-2-infected Cells for Analysis of Gene Expression: Human MRC-5 or Ntera-2 cells (ATCC) were infected With HSV-2 SB5 (ATCC VR-2546) at a multiplicity of infection of 5. At 1, 2, 4, 6, 8, and 17 h post-infection, RNA was isolated by using the TRIzol reagent as described by manufacturer (Life Technologies-Gibco BRL, Grand Island, N.Y.). Mock-infected cells were used as controls in all experiments.

Complementary DNA Preparation from RNA for Hybridization Probes: Twenty ug of total RNA was used to generate Cy3 -labeled cDNA probes (dCTP) using BRL kit 18089-011 (Gibco BRL life Technologies). Probes were purifies using Qiagen Qiaquick PCR columns. Follow manufacturers protocol, except for an additional spin prior to washing.

Complementary DNA preparation from RNA for RT-PCR experiments: RNA was digested with RNase-free DNase I (Boehringer Mannheim Biochemicals, Indianapolis, Ind.) for 45 minutes followed by 5 minutes incubation at 70 degrees C. to inactivate the enzyme. Complementary DNA (50 ul) was generated from 2-3 ug of total RNA using Superscript Preamplification kit (Life Technologies-Gibco BRL, Grand Island, N.Y.) priming with oligo (dT) and random hexamers as described previously (Tal-Singer R., T. M. Lasner, W. Podrzucki, A. Skokotas, J. J. Leary, S. L. Berger, and N. W. Fraser. 1997. Gene expression during reactivation of herpes simplex virus type I from latency in the peripheral nervous system is different from that during lytic infection of tissue cultures. J Virol 71:5268-5276).

PCR amplification of cDNA for Semi-quantitative Analysis: Reactions were performed in 25 ul volumes containing appropriate amounts of cDNA. Primer pairs used to detect SB5 transcripts are described in Table 1. Primers for GAPDH were obtained from Clonetech. Primers for beta actin and cyclophilin were described previously (Tal-Singer R., T. M. Lasner, W. Podrzucki, A. Skokotas, J. J. Leary, S. L. Berger, and N. W. Fraser. 1997. Gene expression during reactivation of herpes simplex virus type 1 from latency in the peripheral nervous system is different from that during lytic infection of tissue cultures. J Virol 71:5268-5276, Tal-Singer R., W. Podrzucki, T. M. Lasner, A. Skokotas, J. J. Leary, N. W. Fraser, and S. L. Berger. 1998. Use of differential display reverse transcription-PCR to reveal cellular changes during stimuli that result in herpes simplex virus type 1 reactivation from latency: upregulation of immediate-early cellular response genes TIS7, interferon, and interferon regulatory factor-1. J Virol 72:1252-1261). Cycling reactions were performed using 1 uM each primer, 1.25 U of AmpliTaq Gold, 200 uM dNTP, and 10×buffer with 25 mM MgCl2 in 96-well plates using thermal cycler 9700 (Perkin-Elmer, Norwalk, Conn.). After one cycle of 9 min. of denaturation of 95° C., cycles were as follows: (i) 1 minute of denaturation at 95° C. (ii) annealing at 60° C. for 1 min, and (iii) extension for 2 min at 72° C. The final cycle was terminated with a 7 min extension at 72° C. Amplification was carried out for 35 to 45 cycles. RNA samples without reverse transcription were included in each set of experiments to control for DNA contamination (RT-). PCR products were analyzed by agarose gel electrophoresis, Fluoimager scanning, (Molecular Dynamics) and band intensity quantitation as described previously (Tal-Singer et al. 1998). The relative amount of PCR product was determined in arbitrary numbers as the ratio between the PCR product band intensity and that of a cellular housekeeping gene, encoding cyclophilin, beta-actin or GAPDH Bloom, D. C., G. B. Devi-Rao, J. M. Hill, J. G. Stevens, and E. K. Wagner. 1994. Molecular analysis of herpes simplex virus type 1 during epinephrine-induced reactivation of latently infected rabbits in vivo. J. Virol. 68:1283-1292.

PCR standards: HSV-2 (SB5) Viral DNA from infected MRC-5 cells was serially diluted in mouse DNA prepared from brains by using DNAzol reagent (Life Technologies-Gibco BRL, Grand Island, N.Y.). A total of 10 nanogram in 1 ul was subjected to PCR with each primer set to evaluate relative primer sensitivity.

Quantitative RNA Analysis by TaqMan : Reactions were performed in 50 ul volumes containing 2×TaqMan Universal PCR Master mix (Perkin-Elmer, Norwalk, Conn.) and appropriate amounts of cDNA. Reactions also contained 200 nM of TaqMan primers and 400 nM of TaqMan probe. Primer pairs and probes described in Table 2 were designed using Primer Express software (Perkin-Elmer, Norwalk, Conn.) and analyzed in 96-well optical plate. Probes were labeled at the 5′ end with the fluorescent reporter dye Fam and at the 3′ end with fluorescent quencher dye Tamra by Synthegen (Houston, Tex.) to allow direct detection of the PCR product. The TaqMan probe hybridizes to a target sequence within the PCR product and cleaves to separate the reporter and quencher dye. The separation of these two dyes increases the fluorescence of the reporter. The resulting fluorescence was measured using ABI 7700 Sequence detector (Perkin-Elmer, Norwalk, Conn.). Relative copy numbers were calculated using a standard curve generated using PCR standards described above.

TABLE 1

Sensitivity of primer pairs used in this study for semi-quantitative
PCR analysis

# of HSV copies detected

| | | | | |
|---|---|---|---|---|
| HSV-2 | Product | 45 Cycles | 35 Cycles | |
| Gene | Size bp | of PCR | of PCR | Forward and Reverse Primer Sequences |

| | | | | |
|---|---|---|---|---|
| LAT | 120 | 100 | 100 | CCAGAAAGGGCAGGCAGGTCAG SEQ ID |
| | | | | NO:1 |
| | | | | GCCGGATCCGCGAAAATAATAACA SEQ ID |
| | | | | NO:9 |
| ICP4 | 111 | 1 | 1000 | GCACGGCGGGCAGCACCTC SEQ ID NO:3 |
| | | | | ACCGCCGCCTCATCGTCGTCAA SEQ ID |
| | | | | NO:4 |
| ICP47 | 101 | 1 | 10 | GATCCTGCCGCTCGTTCG SEQ ID NO:5 |
| | | | | GCTCCCGCTGCTGTGTCCT SEQ ID NO:6 |
| ICP22 | 405 | 1 | 1000 | CGGCGTGCGGGTGTGGTTTTC SEQ ID NO:7 |
| | | | | GGGCTCGGCGGCGGGTTCAA SEQ ID NO:8 |
| ICP27 | 276 | 10 | 10 | GCCCGAGCCTCTACCGCACATT SEQ ID NO:9 |
| | | | | TGGCCGTCAGCTCGCACAC SEQ ID NO:10 |
| UL54B | 522 | 1 | 10 | GCCCGAGCCTCTACCGCACATT SEQ ID |
| | | | | NO:11 |
| | | | | TGGCCGTCAGCTCGCACAC SEQ ID NO:12 |
| ICP6 | 220 | 10 | 100 | CCTCACAGATGCITGACGACGG SEQ ID |
| | | | | NO:13 |
| | | | | GACAGCTCTATCCTGAGT SEQ ID NO:14 |
| gD | 305 | 1 | 10 | CTGGTCATCGGCGGTATT SEQ ID NO:15 |
| | | | | GAGGTGGCTGTGGGCGCG SEQ ID NO:16 |
| gB | 260 | 10 | 100 | CTGGTCAGCTTTCGGTACGA SEQ ID NO:17 |
| | | | | CAGGTCGTGCAGCTGGTTGC SEQ ID NO:18 |
| POL | 305 | 10 | ND | CACTTTCAGAAGCGCAGC SEQ ID NO:19 |
| | | | | ATGTTTGATGCCCGCCAGG SEQ ID NO:20 |
| TK | 124 | 10 | ND | TCCCCGAGCCGATGACTT SEQ ID NO:21 |
| | | | | GTCATTACCGCCGCC SEQ ID NO:22 |
| VP16 | 192 | 1 | 100 | TACGCCGAGCAGATGATG SEQ ID NO:23 |
| | | | | CAGCGGGAGGTFCAGGTG SEQ ID NO:24 |
| gC | 217 | 1 | 10 | CCCGGGGGCCAACTGGTGTATGA SEQ ID |
| | | | | NO:25 |
| | | | | CCGCGTGGGGGTGGATGGTC SEQ ID NO:26 |

TABLE 2

TaqMan primers and probes used in this study for
quantitative analysis

| | | |
|---|---|---|
| HSV-2 | Product Size | |
| Gene | (bp) | Oligonucleotide Sequence |

| | | | |
|---|---|---|---|
| UL9 | F | 67 | GTPAAGACTGTCCGCGA |
| | | | SEQ ID NO:27 |
| | R | | CAGCAAATTCCGGTACAAGC |
| | | | SEQ ID NO:28 |
| | Probe | | CGCCAGCTGCACCTCTCGAA |
| | | | SEQ ID NO:29 |
| ICP27 | F | 54 | TCGAGCGCATCAGCGAA |
| | | | SEQ ID NO:30 |
| | R | | GGCATCCCGCCAAAGG |
| | | | SEQ ID NO:31 |
| | Probe | | ACGCAGTGCCCTGGTCATGCAAC |
| | | | SEQ ID NO:32 |
| ICP6 | F | 67 | CCTCTGGATGCCGGACC |
| | | | SEQ ID NO:33 |
| | R | | CCAGGTGTGACGTTTTTCT |
| | | | SEQ ID NO:34 |
| | Probe | | AAGCGCCTGATCCGCCACCTC |
| | | | SEQ ID NO:35 |
| gC | F | 70 | TTCGATCCGGCCCAGATAC |
| | | | SEQ ID NO:36 |
| | R | | TGGAGACGGTGGAAAAGCC |
| | | | SEQ ID NO:37 |
| | Probe | | CACGCAGACGCAGGAGAACCCC |
| | | | SEQ ID NO:38 |

### Example 1

Identification of Viral Genes Induced During Reactivation.

Genomic viral DNA is prepared from MRC-5 cells infected with strain HSV-2 SB5 (ATCC VR-2546). The DNA is sheared into fragments with an average size of 1 to 2 kb by nebulization and the fragments cloned into pUC19 and Bluescript vectors. Randomly selected, cloned fragments are sequenced from over 2000 individual clones and the sequences are assembled into contiguous DNA sequences representing the HSV-2 genome using Sequencer and PHRAP software. The HSV-2 DNA insert in each clone is amplified by PCR using M13 forward and reverse primers. Five nanograms of each of the PCR product DNA's are then printed as dots onto hundreds of glass slides in duplicate arrays of 25 blocks of 8 rows of dots by 12 columns of dots. Separate aliquots of each PCR product are subjected to one run of DNA sequencing at each end to confirm the linear location of the insert product with the genomic assembly. Control DNA samples, for example from the cellular gene clones from beta-actin, cyclophylin and IRF-1 can be included in the array slides.

Tissues from mice infected with HSV 30 days previously (latently infected mice) are removed before and after induction of reactivation by hypothermia. Tissues collected include brain and trigeminal ganglia. The RNA is purified from the tissues as described in reference 15. Labeled cDNA from latently infected and reactivating tissues will be prepared and hybridized to individual slide arrays of DNA fragments described above. The labeled pattern of dots obtained by hybridizing arrays with cDNA from latently infected animals are compared to the pattern obtained by hybridizing arrays with cDNA from reactivating animals using computer assisted image analysis. The resulting pattern of clones is translated using computer assisted calculations into a linear array of genomic HSV-2 sequences which are hybridized to the RNA's from reactivating tissues. These linear arrays delineate the HSV-2 coding sequences expressed during the reactivation process, and the genes are defined by the first (or in some cases second) ATG 5′ from the end of each RNA predicted from the contiguous linear array. In this example, important genes expressed during reactivation but not during latent infection include the TK gene UL23 and the DNA polymerase gene UL30. Notably, the immediate early genes ICPO, ICP4, and ICP22 are not expressed before the UL23 and UL30 genes as they are during primary infection in vitro, suggesting that a cellular function induced by the hypothermia overcomes or substitutes for transcriptional regulation of UL23 and UL30 by ICPO, ICP4 and ICP22 genes. Thus, antiviral drugs which interfere with ICP0, 4 or 22 would not be expected to interfere with latency as much as inhibitors of UL23 or UL30.

### Example 2

Identification of the Temporal Regulation of Gene Expression in HSV-2 During Primary in Vitro Infection.

The kinetics of the temporal cascade of expression all of the genes in HSV-2 is determined at one time in an experiment employing RNA samples from MRC-5 cells infected with HSV-2 SB5 in vitro for 0, 2, 6, 12 and 18 hours. To more finely determine the end location of RNA transcripts from the internal repeat L to the internal repeat S region, PCR products 1000 bp long starting at every 10 nucleotides between 116,100 to 132,600 are produced and added to the array to supplement the random clones prepared as in Example 1. These new additions guarantee a minimum accuracy of mapping the end of a transcript to within 10 nucleotides of the actual end. Labeled cDNA probes are prepared from the RNA samples prepared 0, 2, 6, 12, and 18 hours after infection with HSV-2. All 5 cDNA probe samples are hybridized to the array grids on glass slides and the pattern of labeled probe binding to spots is again translated into a linear array (or map) of the RNA molecules' template sequence on the HSV-2 genome. In this experiment, no RNA transcripts are detected in the 0 time point, the immediate-early genes including ICP0, ICP4 and ICP22 are detected at the 2 hour time point, and in the 6 hour time point hybridization the early genes including UL23 and UL30 are also detected. By the 18 hour time point, only transcripts representing the structural genes such as glycoprotein D and glycoprotein B are detected. Among the genes detected in each kinetic class are some that are novel, previously unidentified transcripts and transcripts whose HSV-1 homologs are temporally regulated differently than their HSV-2 counterparts.

### Example 3

Identification of the Stage in the HSV Life Cycle at Which a Potential Antiviral Compound Acts, and Clarification of the Mechanism of Action of the Compound.

Since the temporally-regulated cascade of gene expression from HSV-2 can be characterized as in Example 2 above, it follows that the disruption of that cascade can also be determined by fine array transcript mapping through the use of cDNA probes prepared identically except that the infected cells are treated with compound “X”. For example, those genes whose expression is completely dependent upon HSV DNA replication would be identified by hybridizing the arrays to cDNA probes from cultures at 12 to 18 hours after infection in the presence or absence of the DNA synthesis inhibitor aphidicolin. Those genes strictly dependent upon DNA synthesis for their expression would be mapped by the probe from untreated cultures but absent from the mapped transcripts detected through the use of the probe from treated cultures. Subsequently, any compound of unknown activity could be suspected to inhibit HSV DNA synthesis if the same pattern of hybridized dots were detected using cDNA probes from cells 12 to 18 hr after infection in the presence of the unknown compound. Similarly, if only the immediate early genes were detected in mapping with cDNAs from a culture 12 to 18 hours after infection in the presence of an unknown compound, then the compounds mechanism of action would involve and earlier step in the replication cycle, for example the transactivation of gene expression by ICP4.

### Example 4

Identification of Novel Genes Encoded by the HSV Genome.

The temporally-regulated cascade of gene expression from HSV-2 can be characterized as in Example 2 above. Since it is known that there are transcripts from the HSV genomic region around open reading frames UL8, UL9, and UL10 that are of different size than those encoding UL8, UL8.5, UL9, UL9.5 and UL10 (17) and that FAT Mapping will predict the location of the ends of these mRNAs, novel encoded proteins can be predicted.

This prediction will be based on the open reading frame represented by the first ATG codon present in a translation-initiation context from the 5′ end of the alternative RNAs. Some of these RNAs may be expressed rapidly after infection and others later during infection, assisting in separating the signals generated on the cloned DNA spots. The predicted novel proteins may represent a portion of the amino acid sequence of the known UL8, UL8.5, UL9, UL9.5, or UL10 genes (i.e. contain a subsection of those open reading frames), or may represent a new amino acid sequence, by occurring in a different open reading frame.

### Example 5

Characterization of Novel Compounds and Their Drug Potential Through Their Effect on Transcription.

If the genomic sequence subjected to FAT Mapping represents a portion of an animal genome, for example a section of the human genome encoding chemokines, then probes prepared from cells or tissues treated with experimental compounds may be used to identify compounds which effect the expression of the subject chemokines. Thus, human peripheral blood lymphocytes transcribe mRNA's for proinflammatory RANTES, MIP1b and other chemokines upon appropriate stimulation. If the stimulation is then performed in vitro or in vivo in the presence of test compounds, labeled cDNA probes can be prepared from mRNA extracted from those lymphocytes and used to probe the FAT Map array. Probes prepared from cells treated with compounds which inhibit or enhance the production of RANTES or MIP1b mRNAs can be identified by the corresponding decrease or increase in the FAT Map signals. Those compounds which inhibit transcription of RANTES would be potential anti-inflammatory drugs, while those which enhance the production of RANTES would be potential pro-inflammatory drugs.

Similarly, FAT Mapping may be used to characterize the constellation of genes from a given genomic region which are differentially expressed in specific disease situations, e.g. psoriatic skin. If drugs are known or can be identified through FAT Mapping or another transcriptional analysis to differentially affect the expression of those same genes but in the opposite direction (e.g. down rather than up), then a new disease indication for those known drugs may be discovered through FAT Mapping.

### Example 6

Further Embodiments to Example 2

The FATMap technique was used to identify the temporal regulation of HSV-2 gene expression during primary infection of cell cultures. In order to assess whether the microarray FATMap results were indicative of mRNA levels, the same RNA samples were assessed in three additional ways, a) semi-quantitative PCR where amounts of gene-specific products were compared to housekeeping gene products, b) TaqMan real-time quantitative PCR analysis, and c) hybridization signals generated on the same array by multiple spots of DNA from specific genes of HSV-2. In the following, the results for HSV-2 genes ICP6 (UL39) and gC (UL44) by all techniques are shown.

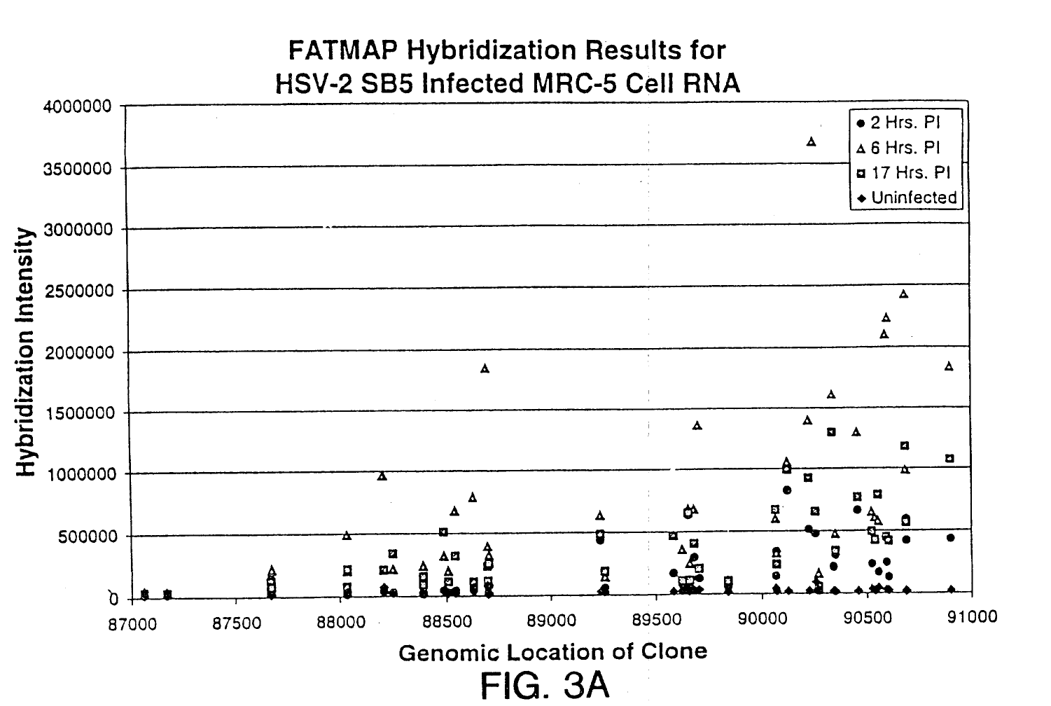

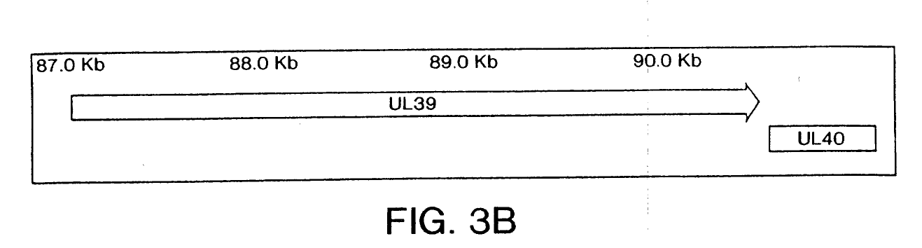

FATMap array hybridization demonstrated a gradual increase of signal for ICP6 (UL39) clones over the time of infection using MRC-5 RNA in comparison to mock infected control cells. The signal intensity peaks at 6 hr PI (post infection) and decreases by 17 hr PI. In FIG. 3A, the image signal intensity is plotted on the Y-axis, while the location of the left end of the clone displaying that signal is used for the X-coordinate. The arrows in FIG. 3B show the open reading frame of UL39 defined in the HSV-2 HG52 genbank entry.

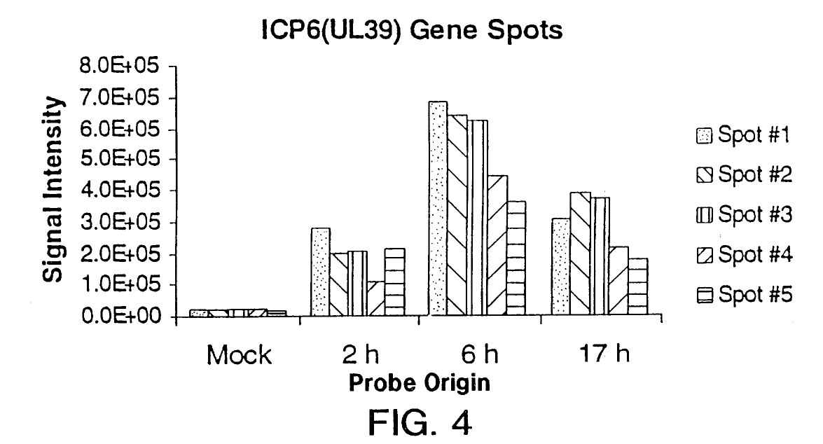

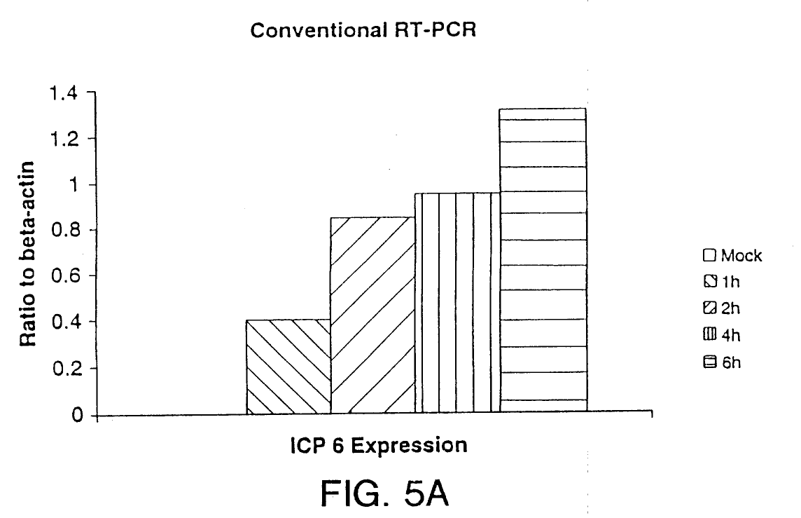

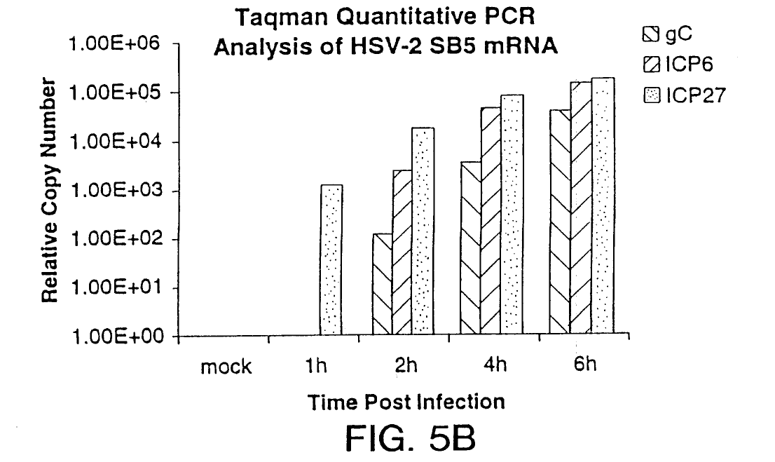

The FATMap data were consistent with the array signals from gene-specific PCR products on the same grid shown in FIG. 4. Conventional semi-quantitative RT-PCR results for the UL39 gene (ICP6) are consistent both with the FATMap array kinetics of expression and the specific gene microarray results, that is an increasing expression up to 6 hr post-infection. Data for conventional RT-PCR with RNA from a similar HSV-2 experiment are shown below in FIG. 5A. The results from the TaqMan quantitative PCR analysis also agreed with the FATMap array in the kinetics of expression of ICP6 (UL39) as shown in FIG. 5B.

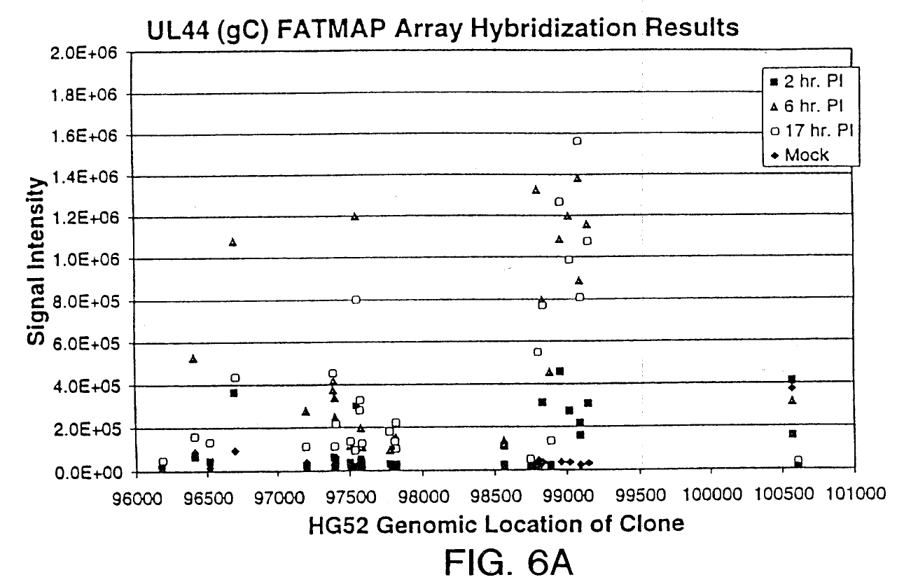

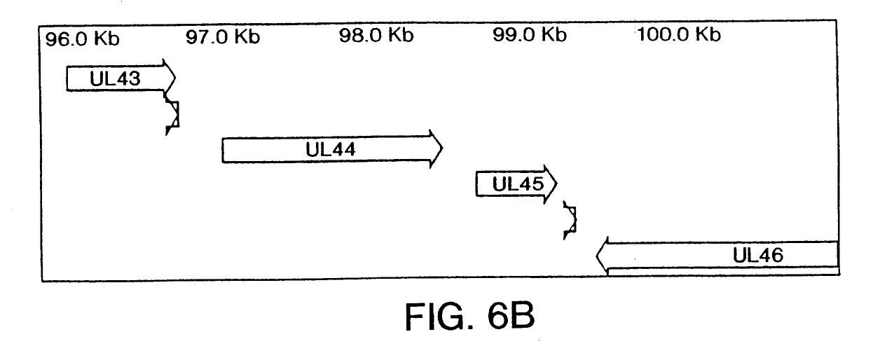

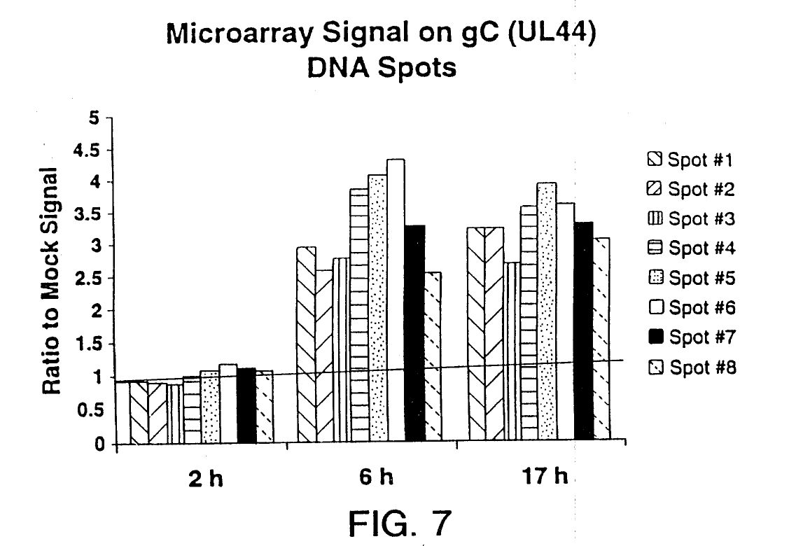

One other HSV-2 gene is included in this example, that being the gene for glycoprotein C, also known as gC, the product of the UL44 open reading frame. In FIG. 6A and 6B, the FATMap data for the UL44 genomic region and the gene map from the HSV-2 HG52 genbank entry are shown. The pattern of expression by FATMap clones above is similar again to the pattern of microarray hybridization done for gene-specific DNA spots for the gC open reading frame (UL44) as shown in FIG. 7. Reproducibility between each of the eight replicate spots of the same UL44 DNA is also good, as shown below.

### Example 7

An Embodiment of Example 4

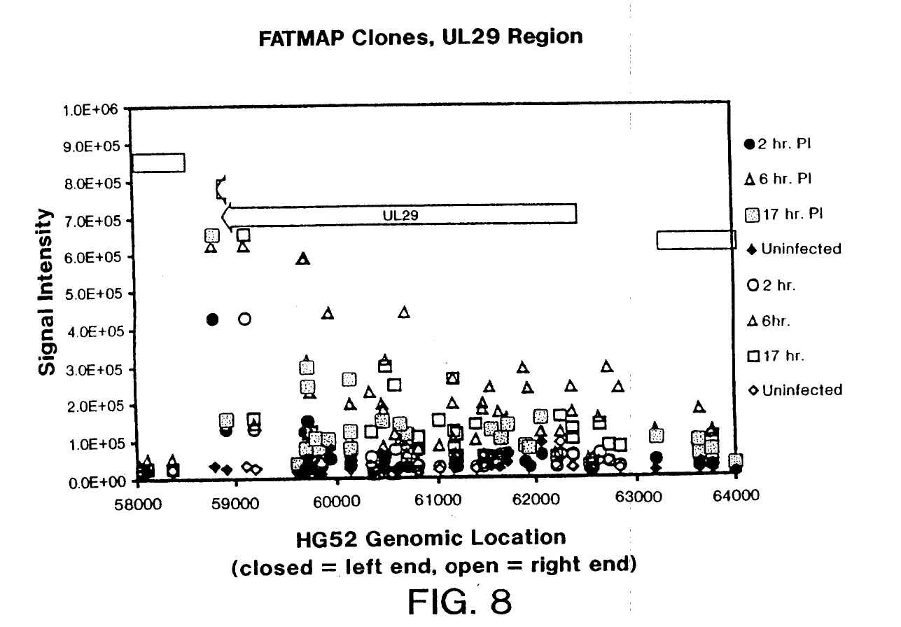

The FATMap technique was used to identify areas of HSV-2 gene expression where the level of expression appears to be different within one open reading frame identified by the HSV-2 HG52 genbank entry. These are cases where it is probably that another RNA exists which does not correlate with the reported genes, and therefore may indicate a new gene. In FIG. 8, below, one can see that the clones spanning the left half of the coding region for UL29 have a much higher signal intensity than those on the right half of the UL29 gene. This suggests a separate, highly expressed RNA, spanning the 3′ half of the gene which conceivably represents expression of a novel gene which uses part of the UL29 open reading frame and one terminus in the UL29 open reading frame. In FIG. 8, the position of both ends of each clone is plotted, with the left end of the clone represented by a filled symbol and the right end of the clone represented by the same symbol, not filled. The height, or Y-axis coordinate of each pair of symbols is the signal intensity shown by the clone in the hybridization experiment.

In FIG. 10, where the clones from the region of UL9 to UL13 are shown, the concept of transcript mapping by FATMap is clearly suggested by the fact that the pattern of clone signals from this region of the genome mimics that of the genes assigned by the HSV-2 HG52 genbank entry. The data point out that UL9 is expressed at low levels, while UL10 and 11 are higher and UL12 is in between in expression level.

References Cited Herein:

The following references are herein incorporated by reference in their entirety into the disclosure of applicants' invention:

1. Chalifur, L. E., R. Fahmy, E. L. Holder, E. W. Hutchinson, C. K. Osterland, H. M. Schipper, and E. Wang. 1994. A method for analysis of gene expression patterns. Analytical Biochemistry. 216:299-304.

2. Diatchenko, L., Y. -F. C. Lau, A. P. Campbell, A. Chenchick, F. Moqadam, B. Huang, S. Lukyanov, K. Lukyanov, N. Gurskaya, E. D. Sverdlov, and P. D. Siebert. 1996. Suppression subtractive hybridization: a method for generating differentially regulated or tissue-specific cDNA probes and libraries. PNAS. 93:6025-6030.

3. Fraser, N. W., J. G. Spivack, Z. Wroblewska, T. Block, S. L. Deshmane, T. Valyi-Nagy, R. Natarajan, and R. Gesser. 1991. A review of the molecular mechanism of HSV-1 latency. Curr. Eye Res. 10 (Suppl):1-14.

4. Fraser, N. W., and T. Valyi-Nagy. 1993. Viral, neuronal and immune factors which may influence herpes simplex virus (HSV) latency and reactivation. Microbial Pathogen. 15:83-91.

5. Hill, T. J. 1985. Herpes simplex virus latency, p. 175-240. In B. Roizman (ed.), The Herpes Viruses, vol. 4. Plenum Publishing Corp., New York.

6. Hubank, M., and D. G. Schatz. 1994. Identifying differences in mRNA expression by representational difference analysis of cDNA. Nucleic Acids Research. 22:5640-5648.

7. Liang, P., D. Bauer, L. Averboukh, P. Warthoe, M. Rohrwild, H. Muller, M. Strauss, and A. B. Pardee. 1995. Analysis of altered gene expression by differential display, p. 304-321. In P. K. Vogt and I. M. Verma (ed.), Methods in Enzymology: Oncogene Techniques, vol. 254. Academic Press, New York.

8. Liang, P., and A. B. Pardee. 1992. Differential Display of eukaryotic messenger RNA by means of the polymerase chain reaction. Science. 257:976-971.

9. Lisitsyn, N., N. Lisitsyn, and M. Wigler. 1993. Cloning the differences between two complex genomes. Science. 259:946-951.

10. Roizman, B. 1991. Herpesviridae: a brief introduction, p. 841-847. In B. N. Fields and D. M. Knipe (ed.), Fundamental virology. Raven Press, Ltd., New York.

11. Schena, M., D. Shalon, R. W. Davis, and P. O. D. Brown. 1995. Quantitative monitoring of gene expression patterns with a complementary DNA microarray. Science. 270:467-470.

12. Shalon, D., S. J. Smith, and P. O. Brown. 1996. A DNA microarray system for analyzing complex DNA samples using two-color fluorescent probe hybridization. Genome-Res. 6:639-45.

13. Sheng, M., and M. E. Greenberg. 1990. The regulation and function of c-fos and other immediate early genes in the nervous system. Neuron. 4:477-485.

14. Stevens, J. G. 1989. Human herpes viruses: A consideration of the latent state. Microbial Rev. 53:318-332.

15. Tal-Singer, R., W. Podrzucki, T. M. Lasner, A. Skokotas, J. J. Leary, N. W. Fraser, and S. L. Berger. 1998. Use of Differential Display-RT-PCR to Reveal Cellular Changes During Stimuli that Result in Herpes Simplex Type 1 Reactivation from Latency: Upregulation of Immediate-Early Cellular Response Genes TIS7, IFN and IRF-1. Journal of Virology, 72: 1252-1261.

16. Winzeler, E. 1997. Functional Genomics of Saccharomyces cerevisiae. ASM news. 63:312-317.

17. Baradaran, K., C. E. Dabrowski, and P. A. Schaffer. 1994. Transcriptional analysis of the region of the herpes simplex virus type 1 genome containing the UL8, UL9, and UL10 genes and identification of a novel delayed-early gene product, OBPC. J. Virol. 68:4251-4261.

18. Jones, K. A., K. R. Yamamoto, and R. Tjian. 1985. Two distinct transcription factors bind to the HSV thymidine kinase promoter in vitro. Cell. 42:559-572.

19. McKnight, S. L. and R. Kingsbury. 1982. Transcriptional control signals of a eukaryotic protein-coding gene. Science. 217:316-324.

20. Loh, E. Y., J. F. Elliott, S. Cwirla, L. L. Lanier, and M. M. Davis. 1989. Polymerase chain reaction with single-sided specificity: analysis of T cell receptor delta chain. Science. 243:217-220.

21. Ohara, O., R. L. Dorit, and W. Gilbert. 1989. One-sided polymerase chain reaction: the amplification of cDNA. Proc. Natl. Acad. Sci. U. S. A. 86:5673-5677.

All publications including, but not limited to, patents and patent applications, cited in this specification or to which this patent application claims priority, are herein incorporated by reference as if each individual publication were specifically and individually indicated to be incorporated by reference herein as though fully set forth.

We claim

1. A method of mapping the position of an individual transcript from a genomic sequence, comprising the steps of:
a) generating overlapping subfragments of the genomic sequence, wherein at least a portion the nucleotide sequence of each genomic subfragment has been determined,
b) placing each overlapping genomic subfragment in a separate ordered (known) position on a high density grid,
c) preparing a composition comprising test transcripts which have been transcribed from said genomic sequence,
d) labeling the test transcripts in said composition in a detectable manner,
e) placing the composition comprising the labeled test transcripts in contact with the high density grid containing the genomic subfragments, whereby the labeled test transcripts are allowed to hybridize to the genomic subfragments,
f) removing unhybridized test transcripts from the surface of the high density grid,
g) detecting on the high density grid the ordered positions which contain a hybridized labeled test transcript, and
h) analyzing the pattern in which the labeled test transcripts have hybridized to the genomic subfragments on the high density grid,
whereby by comparing the position of the labeled test transcripts on the high density grid to the ordered position of the overlapping genomic subfragments on said grid, the position of individual test transcripts from within the genomic sequence are mapped.

2. The method of claim 1 wherein at step a the generation of overlapping subfragments is performed using shotgun cloning techniques.

3. The method of claim 1 wherein the genomic sequence is selected from the group consisting of a plant, animal, bacteria, and a virus.

4. The method of claim 3 wherein the genomic sequence is a human animal.

5. The method of claim 3 wherein the genomic sequence is a herpes virus.

6. The method of claim 1 wherein the overlapping subfragments of step a are amplified using the polymerase chain reaction prior to step b.

7. The method of claim 1 wherein the comparison of the position of labeled test transcripts on the high density grid to the ordered position of the overlapping genomic subfragments on said grid is carried out using computer-assisted methods.

8. The method of claim 1 wherein the individual transcript from the genomic sequence represents transcription of a previously unidentified gene.

9. A method of measuring the differential expression of transcripts between two or more different viral, tissue or cell populations which share a common genomic sequence, comprising the steps of:
conducting the method of claim 1 steps a. and b. on said common genomic sequence;
separately performing the method of claim 1 steps c. through h. on each different viral, tissue or cell population; and
comparing the pattern in which the test transcripts from each different viral, cell or tissue population have been mapped to the common genomic sequence;
whereby differences in the expression of transcripts between the different viral, tissue or cell populations is determined.

10. The method of claim 9 wherein the differential expression of transcripts between two or more tissues within the same organism is measured.

11. The method of claim 9 wherein the differential expression of one viral, cell or tissue population is measured at different time points.

12. The method of claim 9 wherein the differential expression of one tissue, viral or cell population is measured in the absence and presence of an external stimulus or in the absence and presence of a disease state.

13. The method of claim 12 wherein the external stimulus is a chemical compound.

14. The method of claims 11 wherein the viral, tissue or cell population is selected from the group consisting of bacteria and virus.

15. The method of claims 13 and 14 wherein the viral population is herpes virus type 2.

16. The method of claims 11 and 14 wherein the viral population is herpes virus type 2, and time points are taken at various intervals over the course of viral infection, latency and reactivation.

17. A method of determining whether a particular open reading frame of known position within a genomic sequence is expressed under particular conditions, comprising the steps of:
conducting the method of claim 1 steps a. and b. on a genomic sequence, whereby the ordered position on the high density grid of genomic subfragments corresponding to said particular open reading frame is determined,
subjecting a population of viral, cells or tissues containing said genomic sequence to a particular condition;
conducting the method of claim 1 steps c. through h. on the genomic sequence of said cells or tissues which have been subjected to the particular condition; and
determining whether test transcripts from said viral, cells or tissues which have been subjected to said particular condition have hybridized to the ordered positions on said high density grid corresponding the genomic subfragments of said particular open reading frame;
whereby it is determined whether said open reading within said genomic sequence has been expressed under said particular condition.

18. The method of claim 17 wherein the particular condition is introduction of a chemical compound prior to or during transcription.

19. The method of claim 17 wherein the genomic sequence is viral.

20. The method of claims 18 and 19 wherein the genomic sequence is from herpes type 2 and the particular condition is introduction of a chemical compound which is a potential antiviral dug.

21. The portions of the nucleotide sequence of each genomic subfragements determined of step a of claim 1 are only from the 3′ and 5′ ends.

22. The portions of the nucleotide sequence of each genomic subfragements determined of claim 9 are only from the 3′ and 5′ ends.

23. The portions of the nucleotide sequence of each genomic subfragements determined of claim 17 are only from the 3′ and 5′ ends.


SEQUENCE LISTING (38 sequences)

SEQ ID NO: 1 - DNA, 22 bp, Homo sapiens
ccagaaagggcaggcaggtcag

SEQ ID NO: 2 - DNA, 24 bp, Homo sapiens
gccggatccgcgaaaataataaca

SEQ ID NO: 3 - DNA, 19 bp, Homo sapiens
gcacggcgggcagcacctc

SEQ ID NO: 4 - DNA, 22 bp, Homo sapiens
accgccgcctcatcgtcgtcaa

SEQ ID NO: 5 - DNA, 18 bp, Homo sapiens
gatcctgccgctcgttcg

SEQ ID NO: 6 - DNA, 19 bp, Homo sapiens
gctcccgctgctgtgtcct

SEQ ID NO: 7 - DNA, 21 bp, Homo sapiens
cggcgtgcgggtgtggttttc

SEQ ID NO: 8 - DNA, 20 bp, Homo sapiens
gggctcggcggcgggttcaa

SEQ ID NO: 9 - DNA, 22 bp, Homo sapiens
gcccgagcctctaccgcacatt

SEQ ID NO: 10 - DNA, 19 bp, Homo sapiens
tggccgtcagctcgcacac

SEQ ID NO: 11 - DNA, 22 bp, Homo sapiens
gcccgagcctctaccgcacatt

SEQ ID NO: 12 - DNA, 19 bp, Homo sapiens
tggccgtcagctcgcacac

SEQ ID NO: 13 - DNA, 22 bp, Homo sapiens
cctcacagatgcttgacgacgg

SEQ ID NO: 14 - DNA, 18 bp, Homo sapiens
gacagctctatcctgagt

SEQ ID NO: 15 - DNA, 18 bp, Homo sapiens
ctggtcatcggcggtatt

SEQ ID NO: 16 - DNA, 18 bp, Homo sapiens
gaggtggctgtgggcgcg

SEQ ID NO: 17 - DNA, 20 bp, Homo sapiens
ctggtcagctttcggtacga

SEQ ID NO: 18 - DNA, 20 bp, Homo sapiens
caggtcgtgcagctggttgc

SEQ ID NO: 19 - DNA, 18 bp, Homo sapiens
cactttcagaagcgcagc

SEQ ID NO: 20 - DNA, 18 bp, Homo sapiens
atgttgatgcccgccagg

SEQ ID NO: 21 - DNA, 18 bp, Homo sapiens
tccccgagccgatgactt

SEQ ID NO: 22 - DNA, 15 bp, Homo sapiens
gtcattaccgccgcc

SEQ ID NO: 23 - DNA, 18 bp, Homo sapiens
tacgccgagcagatgatg

SEQ ID NO: 24 - DNA, 18 bp, Homo sapiens
cagcgggaggttcaggtg

SEQ ID NO: 25 - DNA, 23 bp, Homo sapiens
cccgggggccaactggtgtatga

SEQ ID NO: 26 - DNA, 20 bp, Homo sapiens
ccgcgtgggggtggatggtc

SEQ ID NO: 27 - DNA, 17 bp, Homo sapiens
gttaagactgtccgcga

SEQ ID NO: 28 - DNA, 20 bp, Homo sapiens
cagcaaattccggtacaagc

SEQ ID NO: 29 - DNA, 20 bp, Homo sapiens
cgccagctgcacctctcgaa

SEQ ID NO: 30 - DNA, 17 bp, Homo sapiens
tcgagcgcatcagcgaa

SEQ ID NO: 31 - DNA, 16 bp, Homo sapiens
ggcatcccgccaaagg

SEQ ID NO: 32 - DNA, 23 bp, Homo sapiens
acgcagtgccctggtcatgcaac

SEQ ID NO: 33 - DNA, 17 bp, Homo sapiens
cctctggatgccggacc

SEQ ID NO: 34 - DNA, 19 bp, Homo sapiens
ccaggtgtgacgtttttct

SEQ ID NO: 35 - DNA, 21 bp, Homo sapiens
aagcgcctgatccgccacctc

SEQ ID NO: 36 - DNA, 19 bp, Homo sapiens
ttcgatccggcccagatac

SEQ ID NO: 37 - DNA, 19 bp, Homo sapiens
tggagacggtggaaaagcc

SEQ ID NO: 38 - DNA, 22 bp, Homo sapiens
cacgcagacgcaggagaacccc